(19) United States
(12) Patent Application Publication
(10) Pub. No.: US 2020/0163588 A1
(43) Pub. Date: May 28, 2020

(54) Systems, Methods, And Devices For Tremor Reduction

(71) Applicants: Nicolette Elizabeth Prevost, Atlanta, GA (US); Nisha Sarah Goddard, Trabuco Canyon, CA (US); Colten Christopher Spivey, Franklin, TN (US)

(72) Inventors: Nicolette Elizabeth Prevost, Atlanta, GA (US); Nisha Sarah Goddard, Trabuco Canyon, CA (US); Colten Christopher Spivey, Franklin, TN (US)

(21) Appl. No.: 16/693,342

(22) Filed: Nov. 24, 2019

Related U.S. Application Data
(60) Provisional application No. 62/771,049, filed on Nov. 24, 2018.

(51) Int. Cl. A61B 5/11; A61B 5/00; G06F 3/01

(52) U.S. Cl. CPC A61B 5/1101; A61B 5/7475; A61B 5/6825; A61B 2562/0219; G06F 3/016; G06F 3/014; G06F 3/017; A61B 5/6824

(57) ABSTRACT

A dynamically adjustable wearable therapeutic system. The system can include a first wearable item worn on a first portion of a body and a second wearable item worn on the second portion of the body. The first wearable item can determine bodily forces associated with first movements of a first portion of a body and the second wearable item can determine bodily forces associated with second movements of a second portion of the body. Also, the system can include the first wearable item applying opposing forces and the second wearable item applying opposing forces to the first portion and the second portion of the body, respectively.

### CROSS-REFERENCE TO RELATED APPLICATION

This Application claims the benefit of, and priority under 35 U.S.C. § 119(e) to, U.S. Provisional Patent Application No. 62/771,049, filed Nov. 24, 2018, the contents of which are hereby incorporated by reference in its entirety as if fully set forth below.

### FIELD OF THE INVENTION

The present invention relates generally to systems, methods, and devices for tremor reduction and, more particularly, to systems, methods, and devices for dynamically applying an opposing force to a tremor to mitigate its impact.

### BACKGROUND

Strokes, traumatic brain injuries, Essential Tremor, dystonia, Multiple Sclerosis, and Parkinson's Disease cause those afflicted to suffer from tremors, which can occur in the arms, hands, torso, and legs. The effects of the tremors severely impair the ability to perform various routine functions such as writing, typing, and even holding dining utensils. Most solutions to tremors typically are geared toward medications, therapy, wearable devices, and/or surgery. While medication and surgery can reduce tremors, they can respectively carry side effects or be invasive. Meanwhile, therapy can be time-consuming and difficult, and can provide limited reduction of tremors. On the other hand, wearable devices can provide reduction of tremors with little to no effort; however, wearable devices are often cumbersome and nonspecific to the patient.

Accordingly, there is a need for improved systems, methods, and devices that provide user-specific dynamic tremor reduction.

### SUMMARY

Aspects of the disclosed technology include systems, methods and devices for tremor reduction. Consistent with the disclosed embodiments, systems include one or more processors, devices, transceivers, accelerometers, microphones, or gyroscopes. One exemplary method includes applying a first sleeve of a first wearable device to a first portion of a body. The first wearable device includes a first accelerometer, a first transceiver, a first gyroscope, a first microphone, and one or more first processors. The first accelerometer can determine a first amount of force associated with first movements of the body that are within a predetermined distance of the first portion of the body. Next, based at least in part on the first amount of force associated with the first movements, the one or more processors can determine a first amount of opposing force. Further, the first accelerometer can determine a direction of the first movements. Then, using the first gyroscope, the method can include applying the first amount of opposing force to the first portion of the body in an opposite direction of the direction of the first movements.

For example, a user can place a first sleeve of the wearable device on his hand (e.g., first portion of the body) to stop his hand from shaking. The wearable device (e.g., first accelerometer) can determine how much force is generated when the user's hand shakes and the direction that the hand is shaking (e.g., downward). Then, the wearable device (e.g. first gyroscope) can apply an amount of opposing force in an opposite direction (e.g., upward) to the hand. By doing so, the wearable device can prevent the user's hand from shaking.

In some embodiments, the method can further include applying a second sleeve of a second wearable device to a second portion of the body. The second wearable device comprises a second accelerometer, a second transceiver, a second microphone, and a second gyroscope. The second wearable device can determine, with the second accelerometer, a second amount of force associated with second movements, wherein the second movements are within a predetermined distance of the second portion of the body. Further, the second wearable device can determine, with the second accelerometer, a direction of the second movements. Then, the second wearable device can determine a second amount of opposing force to be applied to the second portion of the body based at least in part on the second amount of force associated with the second movements. Next, using the second gyroscope, the second wearable device can apply the second amount of opposing force to the second portion of the body in an opposite direction of the direction of the second movements.

Similar to the above example, the user can place cover his elbow (e.g., second portion of the body) with a second sleeve of the wearable device to stop his arm from shaking. The wearable device (e.g., second accelerometer) can determine how much force is generated when the user's arm shakes and the direction that the arm is shaking (e.g., downward). Then, the wearable device (e.g. second gyroscope) can apply an amount of opposing force in an opposite direction (e.g., upward) to the arm. Thus, the wearable device can prevent the user's arm from shaking.

According to some embodiments, determining the first amount of opposing force is further based on the second amount of force associated with the second movements.

In some embodiments, determining the second amount of opposing force is further based on the first amount of force associated with the first movements.

In some embodiments, determining the first amount of opposing force can be further based on determining a first body part closest to the first movements and adjusting the first amount of opposing force based on the first body part.

In some embodiments, determining the second amount of opposing force can be further based on determining a second body part closest to the second movements and adjusting the second amount of opposing force based on the second body part.

According to some embodiments, determining the first amount of opposing force and the second amount of opposing force can further involve identifying a point of origin for the first movements, calculating a total amount of opposing force based on tallying the first amount of opposing force and the second amount of opposing force, determining a first amount of distance between the point of origin and an area associated with the first movements, determining a second amount of distance between the point of origin and an area associated with the second movements, determining a ratio of the first amount of distance to the second amount of distance, and determining the first amount of opposing force and the second amount of opposing force by applying an inverse the ratio to the total amount of opposing force.

Therefore, the wearable device can determine that two areas of the body (e.g., a hand and an elbow) are shaking because of the same tremor. Next, the wearable device determines where the tremor originates, and the total amount of force associated with the tremor at the hand and elbow. Then, the wearable device apportions an opposing amount of force to the hand and elbow based on the respective distance to the origin of the tremor.

In some embodiments, the method can further include receiving a user command from a user, receiving a voice command from a user, and/or receiving a user selection. Based on the user command, the voice command, and/or the user selection, the first amount of opposing force and/or the second amount of opposing force can be adjusted.

Thus, the user can adjust the amount of opposing force applied by the wearable device by either saying an audio command, selecting an option to adjust the amount of opposing force on the wearable device, and/or by selecting an option on a user device that is paired to the wearable device.

An exemplary device includes a wearable therapeutic device comprising a sleeve configured to cover at least a portion of a body. The sleeve can comprise at least one pocket configured to hold one or more objects, and a fastener attached to the at least one pocket, the fastener allowing insertion and removal of the one or more objects into and from the at least one pocket.

In some embodiments, the wearable therapeutic device can further comprise an accelerometer that determines an amount of force associated with movements within a predetermined distance of the at least a portion of the body, and one or more processors that determine an amount of opposing force based at least in part on the amount of force associated the movements.

In some embodiments, the one or more objects can comprise one or more gyroscopes that apply the amount of opposing force to the at least a portion of the body in an opposite direction of the direction of the movements.

According to some embodiments, the sleeve is a glove configured to cover at least a portion of a hand.

In some embodiments, the fastener is positioned on a rear portion of the glove that corresponds to a back of the hand.

In some embodiments, the sleeve comprises a material selected from the group consisting of cotton, silk, polyester, nylon, a lycra blend, a spandex blend, and a combination thereof.

In some embodiments, the material has tensile an elongation in the range of 58% to 75%.

In some embodiments, the fastener can be a member selected from the group consisting of a zipper, buttons, toggles, studs, snap fasteners, poppers, eyelets, velcro, frogging, hooks and eyes, magnets, grommets, brooches, safety pins, fabric ties, laces, and a combination thereof.

According to some embodiments, the one or more objects comprise one or more weights selected from the group consisting of tungsten, steel, aluminum, copper, sand, foam, gel, lead, rubber, and a combination thereof.

In some embodiments, the one or more weights can have a square shape, a rectangular shape, a triangular shape, a thin disk-shape, and/or a ball-like shape.

In some embodiments, the one or more weights can have a total weight of less than two pounds.

In some embodiments, the wearable therapeutic device can be further configured to receive a user selection, a voice command and/or a user command. Based on the user command, the voice command, and/or the user selection, the first amount of opposing force and/or the second amount of opposing force can be adjusted.

Further features of the disclosed design, and the advantages offered thereby, are explained in greater detail hereinafter with reference to specific embodiments illustrated in the accompanying drawings, wherein like elements are indicated be like reference designators.

### BRIEF DESCRIPTION OF THE DRAWINGS

Reference will now be made to the accompanying drawings, which are not necessarily drawn to scale, are incorporated into and constitute a portion of this disclosure, illustrate various implementations and aspects of the disclosed technology, and, together with the description, serve to explain the principles of the disclosed technology. In the drawings:

FIG. 1 is an example system for tremor reduction, in accordance with some examples of the present disclosure;

FIG. 2 is an image depicting a wearable therapeutic device, in accordance with some examples of the present disclosure;

FIG. 3 is an example flow chart of a method for tremor reduction using a wearable device, in accordance with some examples of the present disclosure; and

FIG. 4 is another example flow chart of a method for tremor reduction using a tremor reduction system, in accordance with some examples of the present disclosure.

### DETAILED DESCRIPTION

Some implementations of the disclosed technology will be described more fully with reference to the accompanying drawings. This disclosed technology can be embodied in many different forms, however, and should not be construed as limited to the implementations set forth herein. The components described hereinafter as making up various elements of the disclosed technology are intended to be illustrative and not restrictive. Many suitable components that would perform the same or similar functions as components described herein are intended to be embraced within the scope of the disclosed electronic devices and methods. Such other components not described herein can include, but are not limited to, for example, components developed after development of the disclosed technology.

It is also to be understood that the mention of one or more method steps does not imply that the methods steps must be performed in a particular order or preclude the presence of additional method steps or intervening method steps between the steps expressly identified.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

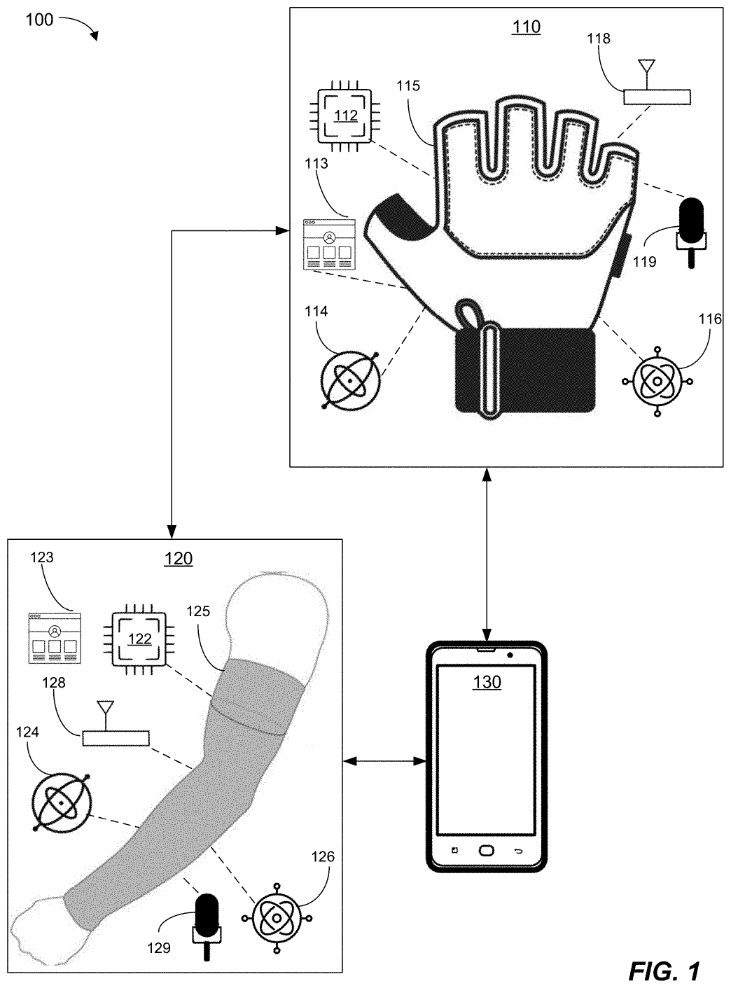

FIG. 1 is a schematic of an exemplary system 100 used for tremor reduction. As shown, the system 100 includes a first wearable device 110, a second wearable device 120, and a user device 130. The first wearable device 110, the second wearable device 120, and the user device 130 communicate with one another. The first wearable device 110 includes one or more first processors 112, a first graphical user interface (GUI) 113, a first accelerometer 114, a first sleeve 115, a first gyroscope 116, a first transceiver 118, and a first microphone 119, among other things. As depicted, the first sleeve 115 can be a fingerless glove that covers part of a hand. Of course, the first sleeve 115 can be any object that covers a portion of the body. The first sleeve 115 (and a second sleeve 125 mentioned below) can be comprised of a material made from cotton, silk, polyester, nylon, a LYCRA blend, a spandex blend, and/or the like. Further, the material can have a tensile elongation in the range of 58% to 75%. Certain portions of the first wearable device 110 can embedded within the first sleeve 115, for example, the one or more first processors 112, the first accelerometer 114, the first gyroscope 116, the first microphone 119, and/or the first transceiver 118. The first GUI 113 can be located at a position on top of the first sleeve 115, such that it is viewable by a user. As illustrated, once the first sleeve 115 is applied to the hand, the first wearable device 110, using the first accelerometer 114, determines a first amount of force associated with first movements within a predetermined distance of the first portion of the body (e.g., hand tremors within seven inches of the wrist). The first wearable device 110 also determines, using the first accelerometer 114, a direction of the first movements (e.g., left, right, up, or down). Next, the first gyroscope 116 applies the first amount of opposing force to the first portion of the body in an opposite direction of the direction of the first movements, such that the first movements (e.g., tremors) are stopped or at least reduced.

Similar to the first wearable device 110, the second wearable device 120 includes one or more second processors 122, a second GUI 123, a second accelerometer 124, a second sleeve 125, a second gyroscope 126, a second transceiver 128, and a second microphone 129, among other things. As shown, the second sleeve 125 can be an elbow sleeve that covers the elbow. Of course, the second sleeve 125 can cover any part of the body, for example, the knee, the ankle, or the wrist. The second sleeve 125 can be comprised of any of the materials listed above in regard to the first sleeve 115, and can also have a tensile elongation in the range of 58% to 75%. The second sleeve 125 can be arranged similar to the first sleeve 115, such that the one or more second processors 122, the second accelerometer 124, the second gyroscope 126, the second transceiver 128, and/or the second microphone 129 are embedded within the second sleeve 125. Of course, akin to the first GUI 113, the second GUI can be located at a position on top of the second sleeve 125.

The second wearable device 120 can use the second accelerometer 124 to determine a second amount of force associated with second movements within a predetermined distance of the second portion of the body (e.g., tremors within five inches of the elbow). Also, the second wearable device 120 can determine, using the second accelerometer 124, a direction of the second movements. Next, the second gyroscope 126 can apply the second amount of opposing force to the second portion of the body in an opposite direction of the direction of the second movements. As a result, the second movements can be stopped or at least reduced. In some embodiments, the first amount and the second amount of opposing force can be based on both the amount of force of the first movements and the second movements. Further, the one or more first processors 112 and/or the one or more second processors 122 can identify a point of origin of both the first movements and the second movements. Next, an amount of distance from an area associated with the first movements to the point of origin can be determined. Also, an amount of distance from an area associated with the second movements to the point of origin can be determined. Then, a ratio of the amount of distance from an area associated with the first movements and the amount of distance from an area associated with the second movements can be determined and an inverse of the ratio can be applied to the total amount of opposing force (a total of the first amount of opposing force and the second amount of opposing force) to determine the first amount of opposing force and the second amount of opposing force. For example, when the area associated with the first movements is ten centimeters from the point of origin and the area associated with the second movement is five centimeters from the point of origin, a ratio of 2:1 is determined. Then, the inverse of the ratio (e.g., 0.5) can be multiplied by the total opposing force to determine the first amount of opposing force and the second amount of opposing force. Therefore, based on the determined point of origin, the first amount of opposing force and the second amount of opposing force can be apportioned, such that, for example, the movements closest to the point of origin can receive a greater amount of opposing force.

Turning the user device 130, the user device 130 can be, for example, a personal computer, a smartphone, a smartwatch, a laptop computer, a tablet, or other computing device. The user device 130 can be paired to the first wearable device 110 and/or the second wearable device 120 through, for example, Bluetooth®, near-field communication (NFC), WiFi, or any other method known in the art. Using the user device 130, a user can send a user command to the first wearable device 110 and/or the second wearable device 120 to adjust the first amount of opposing force and/or the second amount of opposing force. When the user command is received by the first wearable device 110 and/or the second wearable device 120, the first gyroscope 116 and/or the second gyroscope 126, respectively, can apply an adjusted amount of opposing force based on the user command.

Using the GUIs (e.g., the first GUI 113 and/or the second GUI 123) the first wearable device 110 and/or the second wearable device 120 can communicate with one or more processors (e.g., the one or more first processors 112 and/or the one or more second processors 122) after receiving a selection from the user. The user selection can be a request to adjust the first amount of opposing force and/or the second amount of opposing force. For example, after placing the first wearable device on his hand, the user can desire less opposing force. Once the user selects an option to reduce the first amount of opposing force, the one or more first processors 112 can receive instructions that cause it to reduce the amount of opposing force applied by the first gyroscope 116. It should be understood that the first GUI 113 can receive a user selection for the first wearable device 110 and/or the second wearable device 120. Similarly, the second GUI 123 can receive a user selection for the first wearable device 110 and/or the second wearable device 120.

The amount of opposing force can also be adjusted based on a voice command received from the user. Thus, the first microphone 119 and/or the second microphone 129 can receive a voice command from the user that causes the one or more first processors 112 and/or the one or more second processors to instruct the first gyroscope 116 and/or the second gyroscope 126 to adjust the amount of first opposing force and/or the second opposing force, respectively.

Therefore, as disclosed, the wearable devices (first wearable device 110 and second wearable device 120) can operate individually or in tandem. After the amount of opposing force is determined for each wearable device, a respective gyroscope (e.g., first gyroscope 116, second gyroscope 126) can apply the respective amount of opposing force to the respective portion of the body in the direction opposite the movements, such that the tremors (e.g., first movements, second movements) are reduced.

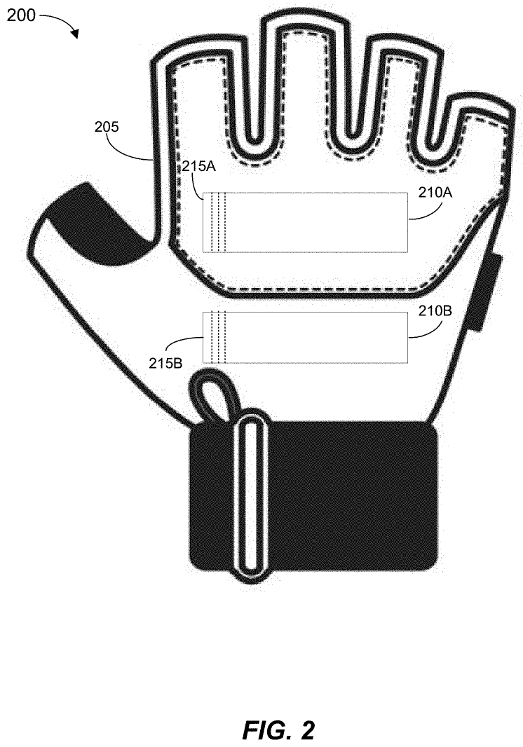

FIG. 2 shows an image of a wearable therapeutic device 200. The wearable therapeutic device 200 includes a sleeve 205 (e.g., a glove) that covers at least a portion of a body (e.g., a hand). The sleeve 205 includes pockets 210A and 210B, which includes fasteners 215A and 215B, respectively. Each pocket 210A and 210B can store one or more objects, for example, an object can be inserted in an open portion of the pocket 210A and then the fastener 215A can be closed to ensure that the object cannot unexpectedly fall out of the pocket 210A. Of course, the pocket 210B can perform the same or similar functions as the pocket 210A. The one or more objects can comprise one or more weights that comprise tungsten, steel, aluminum, copper, sand, foam, gel, lead, and/or rubber. Also, the one or more weights can have a square shape, a rectangular shape, a triangular shape, a thin disk-shape, and/or a ball-like shape. Further, the one or more weights can have a total weight of less than two pounds. In examples where the sleeve 205 is a glove, the fastener 215A and 215B can be positioned on a rear portion of the glove that corresponds to the back of the hand, and the fastener 215A and 215B may be a zipper, buttons, toggles, studs, snap fasteners, poppers, eyelets, velcro, frogging, hooks and eyes, magnets, grommets, brooches, safety pins, and/or or fabric ties and laces. The sleeve 205 can comprise a material made from cotton, silk, polyester, nylon, a lycra blend, and/or a spandex blend. Further, the material can have an elongation in the range of 58% to 75%.

In some embodiments, the wearable therapeutic device 200 can be similar to the first wearable device 110. Thus, the wearable therapeutic device 200 can further include an accelerometer, which can determine an amount of force associated with movements within a predetermined distance of a portion of the body (e.g., the amount of force of movements within five inches of the wrist). The wearable therapeutic device 200 can also include one or more processors (not shown) that determine an amount of opposing force based at least in part on the amount of force associated with the movements. In these embodiments, the one or more objects can comprise one or more gyroscopes that apply the amount of opposing force to the portion of the body in an opposite direction of the direction of the movements. In other words, the one or more gyroscopes can apply an opposing force to the tremors that prevents, in this case, the hand from shaking. Of course, in embodiments where the one or more objects are weights, the weights can provide the opposing force to the portion of the body, such that those tremors are reduced.

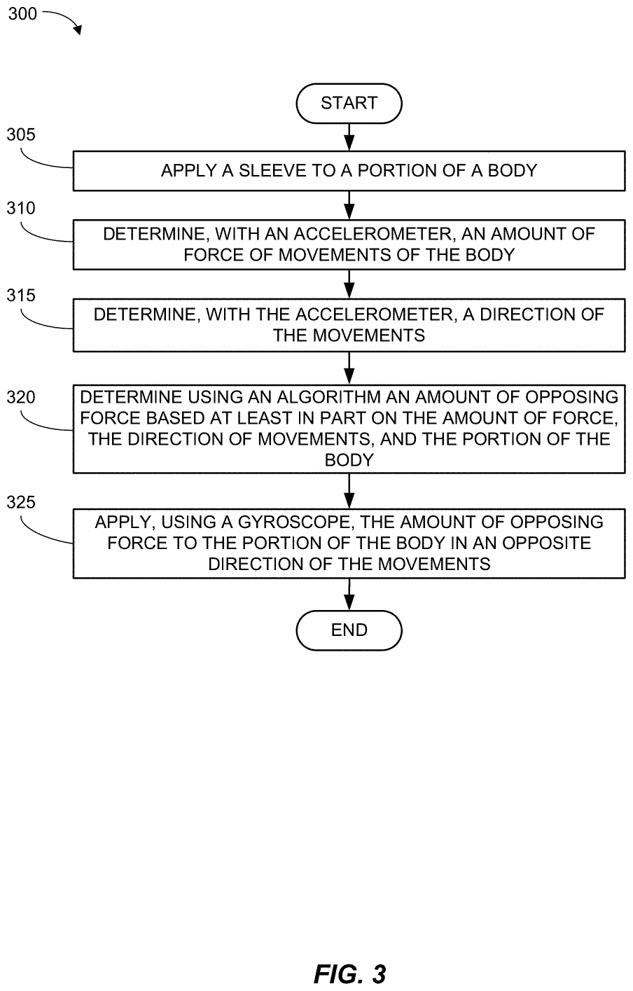

FIG. 3 shows an example flow chart of a method for tremor reduction. The method 300 can performed by the first wearable device 110, the second wearable device 120, and/or the wearable therapeutic device 200. For illustrative purposes only, the method 300 will be described from the perspective of the first wearable device 110.

At 305, the method 300 can include applying the first sleeve 115 (e.g., a wristband) to a portion of the body (e.g., wrist). The first sleeve 115 can include various fasteners (e.g., a zipper, buttons, toggles, studs, snap fasteners, poppers, eyelets, velcro, frogging, hooks and eyes, magnets, grommets, brooches, safety pins, fabric ties and laces, etc.) to provide attachment to the first portion of the body. At 310, the first accelerometer 114 can determine an amount of force of movements within a predetermined distance (e.g., three inches) of the wrist, for example. Next, the first accelerometer 114 can determine the direction of the movements (e.g., upwards and left), at 315.

At 320, based on the amount of force of the movements, the direction of the movements, and/or the portion of the body, the first wearable device 110 can determine an amount of opposing force. The amount of opposing force can be equal to the amount of force of the movements, or it can depend on the portion of the body, for example, the method can also include considering the strength of the muscles, ligaments, and joints of the portion of the body in determining the amount of opposing. As a further example, the wrist may not be able sustain an amount of opposing force comparable to the elbow. As a result, the amount of opposing force can be less than the amount of force of the movements of the body. Also, the direction of the movements can factor into the amount of opposing force, for example, restricting movements of the portion of the body in a typical range of motion can require an amount of opposing force greater than the amount of force of the movements.

At 325, using the first gyroscope 116, the first wearable device 110 can apply the amount of opposing force to the portion of the body in a direction opposite of the movements, such that the movements (tremors) are greatly reduced and/or completely mitigated.

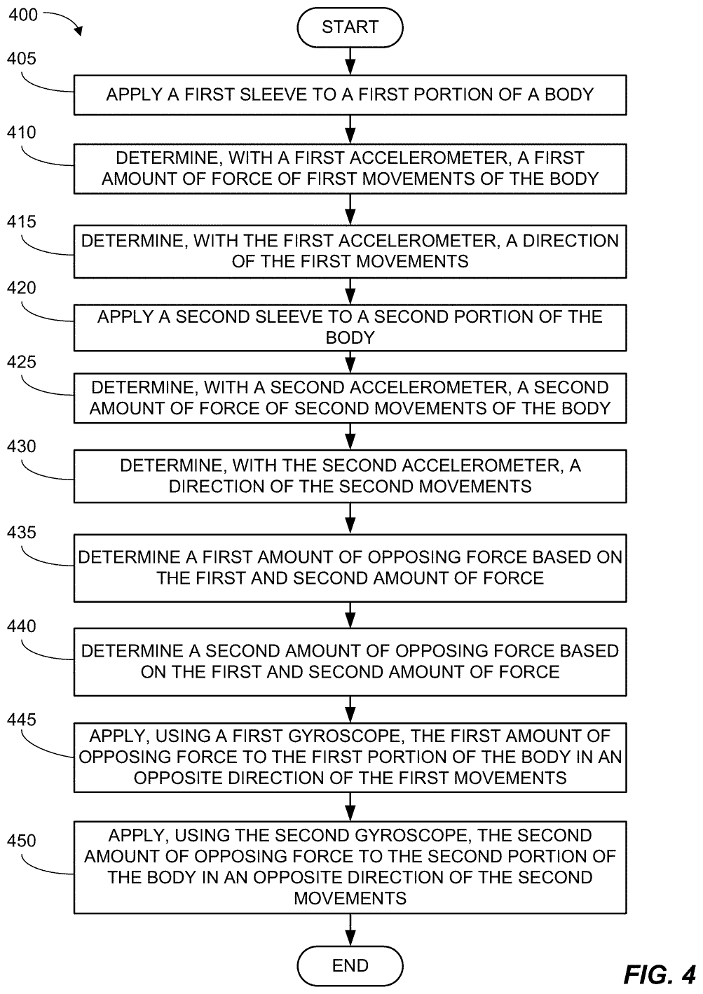

FIG. 4 shows another example flow chart of a method 400 for reducing tremors using a system for tremor reduction (e.g., system 100). Thus, the method 400 can be performed by the first wearable device 110, the second wearable device 120, and/or the user device 130. Further, each of the aforementioned devices may be in communication with one another to perform the method 400.

At 405, the method can include applying the first wearable device 110, and more specifically the first sleeve 115 (e.g., a glove) to a first portion of the body (e.g., a hand). At 410, the first accelerometer 114 can determine the amount of force of the first movements of the first portion of the body (e.g., hand tremors). At 415, the first accelerometer 114 can further determine a direction of the first movements. At 420, by placing the second sleeve 125 (e.g., an elbow sleeve) on a second portion of the body (e.g., the elbow), the second wearable device 120 can be applied to the body. At 425, the second accelerometer 124 can determine a direction of the second movements.

At 430, the second accelerometer 124 can determine the amount of force of the second movements of the second portion of the body (e.g., forearm tremors). At 435, the method can include determining a first amount of opposing force (i.e., an amount of opposing force to be applied in response to the amount of force of the first movements) and, at 440, a second amount of opposing force (i.e., an amount of opposing force to be applied in response to the amount of force of the second movements) based on the amount of force of the first movements and the second movements. In some embodiments, the amount of force of the first movements and the second movements can be further based on the portion of the body where the first sleeve 115 and/or the second sleeve 125 is applied, for example, the strength of the ligaments of the portion of the body can be factored into determining how much opposing force to apply. Also, the method can determine a point of origin of the tremors, and apportion a greater amount of opposing force to the portion of the body that is closer to the point of origin.

At 445, the first wearable device 110, using the first gyroscope 116, can apply the first amount of opposing force to the first portion of the body in an opposite direction of the first movements. Therefore, the first amount of opposing force can reduce or eliminate tremors at the first portion of the body (e.g., the hand). Similarly, at 450, the second wearable device 120, using the second gyroscope 126, can apply the second amount of opposing force to the second portion of the body in an opposite direction of the second movements. Thus, by reducing the tremors (movements) at multiple portions of the body, the system 100 can mitigate the effects of tremors in users.

Throughout the specification and the claims, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

In this description, numerous specific details have been set forth. It is to be understood, however, that implementations of the disclosed technology can be practiced without these specific details. In other instances, well-known methods, structures and techniques have not been shown in detail in order not to obscure an understanding of this description. References to “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described can include a particular feature, structure, or characteristic, but not every implementation necessarily includes the particular feature, structure, or characteristic. Further, repeated use of the phrase “in one implementation” does not necessarily refer to the same implementation, although it can.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the claims, and can include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A wearable therapeutic system comprising:
a wearable item configured to be worn on at least a portion of a body, the wearable item comprising:
a pocket configured to maintain an object therein; and
a fastener having an open condition and a closed condition, the open condition of the fastener configured to allow insertion of the object into the pocket and removal of the object from the pocket, and the closed condition of the fastener configured to secure the object in the pocket.

2. The wearable therapeutic system of claim 1 further comprising:
an accelerometer configured to determine a bodily force associated with a movement of the wearable item; and
a processor storing instructions that when executed, is configured to determine an opposing force based at least in part on the bodily force;
wherein the object comprises a gyroscope configured to apply the opposing force to the wearable item in response to the bodily force.

3. The wearable therapeutic system of claim 2, wherein the processor is further configured to receive, from a user device, a user command to adjust the opposing force into an adjusted opposing force.

4. The wearable therapeutic system of claim 3, wherein the bodily force is a tremor movement; and
wherein the adjusted opposing force is closer to opposite of magnitude and direction of the bodily force than the opposing force is opposite of the magnitude and direction of the bodily force.

5. A dynamic therapeutic system comprising:
a first wearable item configured to:
be worn on at least a first portion of a body; and
move with a first set of movements of the first portion of the body upon which the first wearable item is worn;
a first accelerometer configured to determine in real time varying bodily forces experienced by the first wearable item during the first set of movements; and
a first opposing force generator configured to apply in real time varying opposing forces generally opposing the varying bodily forces so the dynamic therapeutic system provides real time, dynamic dampening of the bodily forces experienced by the first wearable item in an effort to maintain the first wearable item in a still configuration during the first set of movements.

6. The dynamic therapeutic system of claim 5 further comprising a user command configured to adjust at least a portion of the varying opposing forces of the first opposing force generator into adjusted opposing forces;
wherein the adjusted opposing forces maintain the first wearable item more still than the unadjusted opposing forces.

7. The dynamic therapeutic system of claim 6 further comprising a user device configured to receive user input and send the user command.

8. The dynamic therapeutic system of claim 5, wherein each bodily force is a vector having a magnitude and direction;
wherein each opposing force is a vector having a magnitude and direction;
wherein the first set of movements of the first wearable item include tremor movements of the first portion of the body upon which the first wearable item is worn; and
wherein the first opposing force generator comprises a first gyroscope configured to generate the varying opposing forces.

9. The dynamic therapeutic system of claim 8, wherein the first wearable item comprises:
a pocket configured to maintain first opposing force generator therein; and
a fastener having an open condition and a closed condition, the open condition of the fastener configured to allow insertion of the first opposing force generator into the pocket and removal of the first opposing force generator from the pocket, and the closed condition of the fastener configured to secure the first opposing force generator in the pocket.

10. The dynamic therapeutic system of claim 5 further comprising:
a second wearable item configured to:
be worn on at least a second portion of the body different than the first portion; and
move with a second set of movements of the second portion of the body upon which the second wearable item is worn; and
a second accelerometer configured to determine in real time varying bodily forces experienced by the second wearable item during the second set of movements;
wherein the first opposing force generator is configured to apply in real time varying opposing forces at one or both the first wearable item and the second wearable item such that the real time varying opposing forces generally oppose the combination of the varying bodily forces experienced by the first wearable item and the second wearable item so the dynamic therapeutic system provides real time, dynamic dampening of the bodily forces in an effort to maintain one or both the first wearable item and the second wearable item in a still configuration during the first set of movements and the second set of movements.

11. The dynamic therapeutic system of claim 10, wherein the second wearable item comprises:
a pocket configured to maintain second opposing force generator therein; and
a fastener having an open condition and a closed condition, the open condition of the fastener configured to allow insertion of the second opposing force generator into the pocket and removal of the second opposing force generator from the pocket, and the closed condition of the fastener configured to secure the second opposing force generator in the pocket.

12. The dynamic therapeutic system of claim 10, wherein the first opposing force generator is configured to apply in real time varying opposing forces at the first wearable item such that the real time varying opposing forces generally oppose the combination of the varying bodily forces experience by the first wearable item and the second wearable item so the dynamic therapeutic system provides real time, dynamic dampening of the effect of the combined bodily forces in an effort to maintain the first wearable item in a still configuration during the first set of movements and the second set of movements.

13. The dynamic therapeutic system of claim 5 further comprising:
a second wearable item configured to:
be worn on at least a second portion of the body different than the first portion; and
move with a second set of movements of the second portion of the body upon which the second wearable item is worn; and
a second accelerometer configured to determine in real time varying bodily forces experienced by the second wearable item during the second set of movements; and
a second opposing force generator configured to apply in real time varying opposing forces generally opposing the varying bodily forces so the dynamic therapeutic system provides real time, dynamic dampening of the bodily forces experienced by the second wearable item in an effort to maintain the second wearable item in a still configuration during the second set of movements.

14. The dynamic therapeutic system of claim 13, wherein each bodily force is a vector having a magnitude and direction;
wherein each opposing force is a vector having a magnitude and direction;
wherein the second set of movements of the second wearable item include tremor movements of the second portion of the body upon which the second wearable item is worn; and
wherein the second opposing force generator comprises a second gyroscope configured to generate the varying opposing forces.

15. The dynamic therapeutic system of claim 5 further comprising:
a second wearable item configured to:
be worn on at least a second portion of the body different than the first portion; and
move with a second set of movements of the second portion of the body upon which the second wearable item is worn; and
a second accelerometer configured to determine in real time varying bodily forces experienced by the second wearable item during the second set of movements;
a second opposing force generator configured to apply in real time varying opposing forces generally opposing the varying bodily forces so the dynamic therapeutic system provides real time, dynamic dampening of the bodily forces experienced by the second wearable item in an effort to maintain the second wearable item in a still configuration during the second set of movements; and
a user device configured to receive user input and send a user command configured to adjust at least a portion of the varying opposing forces of one or both the first opposing force generator and the second force generator into adjusted opposing forces;
wherein the first wearable item comprises:
a pocket configured to maintain first opposing force generator therein; and
a fastener having an open condition and a closed condition, the open condition of the fastener configured to allow insertion of the first opposing force generator into the pocket and removal of the first opposing force generator from the pocket, and the closed condition of the fastener configured to secure the first opposing force generator in the pocket;
wherein the second wearable item comprises:
a pocket configured to maintain second opposing force generator therein; and
a fastener having an open condition and a closed condition, the open condition of the fastener configured to allow insertion of the second opposing force generator into the pocket and removal of the second opposing force generator from the pocket, and the closed condition of the fastener configured to secure the second opposing force generator in the pocket; and
wherein the first opposing force generator and the second opposing force generator are configured to work together, and apply in real time varying opposing forces at one or both the first wearable item and the second wearable item such that the real time varying opposing forces generally oppose the combination of the varying bodily forces experienced by one or both the first wearable item and the second wearable item so the dynamic therapeutic system provides real time, dynamic dampening of the bodily forces in an effort to maintain one or both the first wearable item and the second wearable item in a still configuration during the first set of movements and the second set of movements; and
wherein the adjusted opposing forces maintain one or both the first wearable item and the second wearable item more still than the unadjusted opposing forces.

16. A method for tremor reduction comprising:
determining, with a first accelerometer of a first wearable item located at a first position of a body, real time varying bodily forces experienced by the first wearable item during a first set of movements;
determining, with a second accelerometer of a second wearable item located at a second position of the body different than the first position, real time varying bodily forces experienced by the second wearable item during a second set of movements;
applying, with a first opposing force generator, real time varying opposing forces generally opposing the varying bodily forces experienced by the first wearable item; and
applying, with a second opposing force generator, real time varying opposing forces generally opposing the varying bodily forces experienced by the second wearable item;
wherein the method provides dynamic dampening of the bodily forces experienced by the first wearable item and the second wearable item in an effort to maintain the first wearable item and the second wearable item in still configurations during the first and second sets of movements.

17. The method of claim 16, wherein the first opposing force generator comprises a first gyroscope; and
wherein the second opposing force generator comprises a second gyroscope.

18. The method of claim 16, further comprising:
in response to receiving a user command from a user device, adjusting at least a portion of the varying opposing forces into adjusted opposing forces;
wherein the adjusted opposing forces maintain the first wearable item and the second wearable item more still than the unadjusted opposing forces.

19. The method of claim 16, wherein each bodily force is a vector having a magnitude and direction;
wherein each opposing force is a vector having a magnitude and direction;
wherein the first set of movements of the first wearable item include tremor movements of the first portion of the body upon which the first wearable item is worn; and
wherein the second set of movements of the second wearable item include tremor movements of the second portion of the body upon which the second wearable item is worn.

20. The method of claim 16, further comprising:
determining the varying opposing forces based on a combination of the varying bodily forces experienced by the first wearable item during a first set of movements and the varying bodily forces experienced by the first wearable item during a first set of movements.
